(19) United States
(12) Patent Application Publication
(10) Pub. No.: US 2026/0151579 A1
(43) Pub. Date: Jun. 4, 2026

(54) CARDIOPULMONARY RESUSCITATION AIRWAY PRESSURE AUTOMATIC FEEDBACK VENTILATION METHOD AND VENTILATOR

(71) Applicant: GUANGZHOU LANDSWICK MEDICAL TECHNOLOGIES LTD., Guangzhou (CN)

(72) Inventors: Hui DONG, Guangzhou (CN); Shengxiang LIN, Guangzhou (CN)

(21) Appl. No.: 19/457,209

(22) Filed: Jan. 23, 2026

Related U.S. Application Data
(63) Continuation of application No. PCT/CN2024/089230, filed on Apr. 23, 2024.

(30) Foreign Application Priority Data
Sep. 14, 2023 (CN) 202311189234.0

(51) Int. Cl. A61M 16/00; A61H 31/00; G16H 20/40

(52) U.S. Cl. CPC A61M 16/024; A61H 31/006; G16H 20/40; A61M 2016/0027; A61M 2016/0033; A61M 2205/05; A61M 2205/332; A61M 2205/702; A61M 2230/04

(57) ABSTRACT

A cardiopulmonary resuscitation airway pressure automatic feedback ventilation method and a ventilator. The method includes: obtaining the current airway pressure signal when compressing the heart of the target individual at a preset sampling frequency; dynamically correcting the current airway pressure signal based on body parameters of the target individual to obtain the target airway pressure signal; continuously analyzing the target airway pressure signal to obtain the compression frequency, compression phase, and signal information for the target individual; training a machine learning model with preset compression parameters and ventilation rule parameters; inputting the compression frequency, compression phase, and signal information for the target individual, and mode setting parameters of the ventilation equipment, into the machine learning model to determine the ventilation probability and ventilation time window of the target airway pressure signal, and controlling the ventilation equipment to perform automatic ventilation on the target individual according to the ventilation time window.

### CROSS-REFERENCE TO RELATED APPLICATION

This application is a continuation of International Patent Application No. PCT/CN 2024/089230, filed Apr. 23, 2024, which claims the priority of Chinese Patent Application No. 202311189234.0, filed Sep. 14, 2023, both of which are herein incorporated by reference in their entirety.

### TECHNICAL FIELD

The present disclosure relates to the field of medical emergency technology, particularly to a cardiopulmonary resuscitation airway pressure automatic feedback ventilation method and a ventilator.

### BACKGROUND

Currently, cardiopulmonary resuscitation (CPR) is the basic and the foremost medical method for rescuing patients with cardiac arrest, with external chest compressions combined with forced ventilation often being the emergency measures that such patients require promptly to maintain basic blood circulation, heartbeat, and respiratory recovery. Existing cardiopulmonary resuscitation typically includes two major components: cardiac compressions and ventilation (breathing); cardiac compressions usually have two forms, manual and mechanical (equipment), with manual compressions used when conditions do not permit, and mechanical (equipment) compressions used in hospitals when conditions allow. Clinical requirements for compressions usually follow: effective external chest compression frequency at 100˜120 times/minute, depth at 5˜6 centimeters; ventilation (breathing) during cardiopulmonary resuscitation emergency is usually performed using: manual blowing, bag-valve-mask ventilation, and ventilator ventilation. Ventilation requirements according to clinical demands include 2 ventilations in the middle of 30 compressions, 2 ventilations after 15 compressions, and continuous uninterrupted compressions with 1 ventilation every 10 compressions. The coordination of existing cardiopulmonary resuscitation compressions and ventilation is usually managed by medical staff or by the coordination of cardiopulmonary resuscitation compression equipment and ventilators through communication protocols. There are issues with the inability to perform automatic coordination and the difficulty of manual coordination methods to precisely and stably control for long periods, reducing practicality.

### SUMMARY

In view of the problems presented above, the present disclosure provides a cardiopulmonary resuscitation airway pressure automatic feedback ventilation method and ventilator to solve the issues mentioned in the background art regarding the coordination of existing cardiopulmonary resuscitation compressions and ventilation, which is usually managed by medical staff or through communication protocols between cardiopulmonary resuscitation compression equipment and ventilators. The inability to perform automatic coordination and the difficulty of manual coordination methods to precisely and stably control for long periods, reducing practicality.

A cardiopulmonary resuscitation airway pressure automatic feedback ventilation method, comprising the following steps:
obtaining the current airway pressure signal when compressing the heart of the target individual at a preset sampling frequency;
dynamically correcting the current airway pressure signal based on the body parameters of the target individual to obtain the target airway pressure signal;
continuously analyzing the target airway pressure signal to obtain the compression frequency, compression phase, and high-dimensional signal information for the target individual;
training a machine learning model with preset compression parameters and ventilation rule parameters; and
inputting the compression frequency, compression phase, and high-dimensional signal information for the target individual, along with the mode setting parameters of the ventilation equipment, into the machine learning model to determine the target pressure signal's ventilation probability and ventilation time window, and controlling the ventilation equipment to perform automatic ventilation on the target individual according to the ventilation time window.

In an embodiment, the dynamic correction of the current airway pressure signal based on the body parameters of the target individual to obtain the target airway pressure signal includes:
detecting the compression force and speed when compressing the heart of the target individual;
setting the preset sampling frequency based on the compression force and speed, identifying the area around the mouth and nose of the target individual as the sampling area;
performing air pressure sampling on the target individual within the sampling area at the preset sampling frequency using a pressure sensor; and
obtaining the current airway pressure signal when compressing the heart of the target individual based on the sampling results.

In an embodiment, before continuously analyzing the current airway pressure signal to obtain the compression frequency, compression phase, and high-dimensional signal information for the target individual, it further includes:
confirming the current airway pressure signal as the initial signal;
using a low-pass filter to remove high-frequency noise signals and other spurious signals from the initial signal, obtaining the first processed initial signal;
eliminating the baseline shift signal from the first processed initial signal by removing the direct current signal, obtaining the second processed initial signal; and
generating a clean proximal pressure signal based on the second processed initial signal.

In an embodiment, the dynamic correction of the current airway pressure signal based on the body parameters of the target individual to obtain the target airway pressure signal includes:
determining the current thoracic resonance parameters of the target individual based on their body parameters;
determining the dynamic correction factor based on the current thoracic resonance parameters and the preset collection parameters of the current airway pressure signal;
using the dynamic correction factor to dynamically correct the current airway pressure signal; and
obtaining the target airway pressure signal based on the correction results.

In an embodiment, continuously analyzing the current airway pressure signal to obtain the compression frequency, compression phase, and high-dimensional signal information for the target individual includes:
analyzing the proximal pressure signal using a time-domain analysis algorithm to obtain the first analysis result;
analyzing the proximal pressure signal using a frequency-domain analysis algorithm to obtain the second analysis result;
determining the compression frequency, compression interval, and duration of a single compression for the target individual based on the first analysis result, and determining the compression phase based on the compression interval and duration of a single compression; and
obtaining the frequency-domain characteristics of the signal based on the second analysis result, and performing a deep analysis of the signal frequency-domain characteristics to obtain high-dimensional signal information.

In an embodiment, the analysis of the proximal pressure signal using a frequency-domain analysis algorithm to obtain the second analysis result includes:
using a window function and Fourier transform on the proximal pressure signal to perform a time-frequency conversion to obtain a spectral signal;
marking the peak of the spectral signal, capturing the area around the peak, as well as the areas of the low-frequency and high-frequency regions;
obtaining the regional signals corresponding to the area around the peak, and the areas of the low-frequency and high-frequency regions; and
performing geometric calculations on the regional signals corresponding to the area around the peak, and the areas of the low-frequency and high-frequency regions using a frequency-domain analysis algorithm to obtain the second analysis result.

In an embodiment, training the machine learning model with preset compression parameters and ventilation rule parameters includes:
generating compression pattern parameters based on a preset machine learning algorithm according to preset compression parameters;
generating the first training sample based on the compression pattern parameters, setting model learning parameters and model output parameters based on ventilation rule parameters; and
training the machine learning model with the first training sample based on the model learning parameters and model output parameters.

In an embodiment, the input of the target individual's compression frequency, compression phase, and high-dimensional signal information, along with the mode setting parameters of the ventilation equipment into the machine learning model to determine the target pressure signal's ventilation probability and ventilation time window, and controlling the ventilation equipment to perform automatic ventilation on the target individual according to the ventilation time window, includes:
obtaining the ventilation compression ratio, target frequency, and ventilation threshold for different ventilation modes based on the mode setting parameters of the ventilation equipment;
determining the target ventilation mode based on the target individual's compression frequency and the different modes'ventilation compression ratio, target frequency, and ventilation threshold;
acquiring the ventilation compression phase under the target ventilation mode, and using a dynamic threshold method to match the ventilation compression phase based on the target individual's compression phase;
determining the ventilation probability of the target pressure signal based on the matching results, and determining the ventilation time window threshold based on the relationship between the position of the ventilation compression phase and the target individual's compression phase; and
controlling the ventilation equipment to perform automatic ventilation on the target individual according to the ventilation time window threshold and the ventilation threshold.

In an embodiment, determining the dynamic correction factor based on the current thoracic resonance parameters and the preset collection parameters of the current airway pressure signal includes:
determining the set chest compression variation amplitude parameters based on the preset collection parameters of the current airway pressure signal;
determining the airway resistance index based on the chest compression variation amplitude parameters and the current thoracic resonance parameters;
generating a respiratory signal sample set using a respiratory mechanics model based on the airway resistance index;
determining the morphological parameter values at various points in the target individual's airway based on the respiratory signal sample set;
determining the range of the stress tensor in the target individual's airway based on the morphological parameter values at various points in the airway;
acquiring characteristic parameters that maintain the steady-state performance of the target individual's airway, and obtaining the airway performance indicators of the target individual based on the characteristic parameters;
determining the stress differentiation in the target individual's airway based on the airway performance indicators and the range of the airway's stress tensor;
determining the standard data for the target individual's chest compression force and the error range of actual detection data based on the stress differentiation; and
determining the dynamic correction factor for the current airway pressure signal based on the standard data for the target individual's chest compression force, the error range of actual detection data, and the detected pressure data corresponding to the current airway pressure signal.

A ventilator suitable for the cardiopulmonary resuscitation airway pressure automatic feedback ventilation method includes: a ventilator body, a ventilation circuit, airflow and pressure detection equipment, a ventilation mask, and a pressure plate;

The pressure plate is used to perform artificial or mechanical chest compressions on the target individual;

The ventilation mask is used to cover the mouth and nose area of the target individual;

The airflow and pressure detection equipment is used to detect the airway pressure signal of the target individual's heart during compression;

The ventilator body is used to analyze the airway pressure signal, obtaining information about the target individual's cardiopulmonary resuscitation compressions, and based on this information, determining the ventilation time window, issuing ventilation commands, and performing ventilation operations;

The ventilation circuit is used to channel the ventilating agent from the ventilator body into the mouth and nose of the target individual.

Other features and advantages of this invention will be described in the subsequent specification, and will, in part, be apparent from the specification, or may be learned by practice of the invention. The objectives and other advantages of the present disclosure may be realized and obtained by means of the structures particularly pointed out in the written specification and drawings.

The following detailed description of the embodiments of the present disclosure will be made with reference to the accompanying drawings.

### BRIEF DESCRIPTION OF DRAWINGS

The drawings are intended to provide further understanding of the present disclosure and constitute a part of this specification. They illustrate embodiments of the present disclosure and, together with the description, serve to explain the principles of the disclosure and are not intended to limit the scope of the disclosure.

FIG. 1 is a workflow diagram of a cardiopulmonary resuscitation airway pressure automatic feedback ventilation method provided by the present disclosure;

FIG. 2 is another workflow diagram of a cardiopulmonary resuscitation airway pressure automatic feedback ventilation method provided by the present disclosure;

FIG. 3 is yet another workflow diagram of a cardiopulmonary resuscitation airway pressure automatic feedback ventilation method provided by the present disclosure;

FIG. 4 is a schematic structural diagram of a ventilator provided by the present disclosure;

FIG. 5 is a screenshot of an embodiment generated according to a cardiopulmonary resuscitation airway pressure automatic feedback ventilation method provided by the present disclosure;

FIG. 6 is a curve of airway pressure during the cardiac compression process;

FIG. 7 is a schematic diagram for signal preprocessing;

FIG. 8 is a schematic diagram for frequency domain analysis of the signal;

FIG. 9 is a schematic diagram for time domain analysis of the signal;

FIG. 10 is a schematic diagram for determining the timing of ventilation using a dynamic threshold method on machine compression signals;

FIG. 11 is a schematic diagram for determining the timing of ventilation using a dynamic threshold method on manual compression signals.

### DETAILED DESCRIPTION OF EMBODIMENTS

The exemplary embodiments will be described in detail here, with examples illustrated in the accompanying drawings. When the following description refers to the drawings, unless otherwise indicated, the same numbers in different drawings refer to the same or similar elements. The embodiments described in the following exemplary embodiments do not represent all embodiments consistent with the present disclosure. Instead, they are merely examples of devices and methods consistent with some aspects of the disclosure as detailed in the appended claims.

Currently, cardiopulmonary resuscitation (CPR) is the basic and foremost medical method for rescuing patients with cardiac arrest. External chest compressions combined with forced ventilation are often the emergency measures that such patients require promptly to maintain basic blood circulation, heartbeat, and respiratory recovery. Existing cardiopulmonary resuscitation typically includes two major components: cardiac compressions and ventilation (breathing); cardiac compressions usually have two forms, manual and mechanical (equipment), with manual compressions used when conditions do not permit, and mechanical (equipment) compressions used in hospitals when conditions allow. Clinical requirements for compressions usually follow: effective external chest compression frequency at 100˜120 times/minute, depth at 5˜6 centimeters; ventilation (breathing) during cardiopulmonary resuscitation emergency is usually performed using: manual blowing, bag-valve-mask ventilation, and ventilator ventilation. Ventilation requirements according to clinical demands include 2 ventilations in the middle of 30 compressions, 2 ventilations after 15 compressions, and continuous uninterrupted compressions with 1 ventilation every 10 compressions. The coordination of existing cardiopulmonary resuscitation compressions and ventilation is usually managed by medical staff or by the coordination of cardiopulmonary resuscitation compression equipment and ventilators through communication protocols. There are issues with the inability to perform automatic coordination and the difficulty of manual coordination methods to precisely and stably control for long periods, reducing practicality. To solve the aforementioned problems, this embodiment discloses a cardiopulmonary resuscitation airway pressure automatic feedback ventilation method.

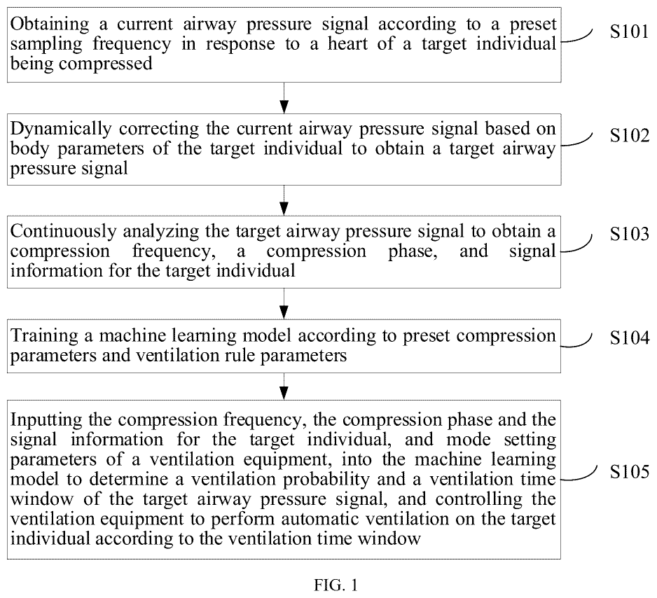

A cardiopulmonary resuscitation airway pressure automatic feedback ventilation method, as shown in FIG. 1, includes the following steps:
step S101, obtaining the current airway pressure signal according to a preset sampling frequency in response to the heart of the target individual being compressed;
step S102, dynamically correcting the current airway pressure signal based on the body parameters of the target individual to obtain the target airway pressure signal;
step S103, continuously analyzing the target airway pressure signal to obtain the compression frequency, compression phase, and high-dimensional signal information for the target individual;
step S104, training a machine learning model according to preset compression parameters and ventilation rule parameters; and
step S105, inputting the compression frequency, compression phase, and high-dimensional signal information for the target individual, along with the mode setting parameters of the ventilation equipment, into the machine learning model to determine the target airway pressure signal's ventilation probability and ventilation time window, and controlling the ventilation equipment to perform automatic ventilation on the target individual according to the ventilation time window and the ventilation probability.

In this embodiment, the preset sampling frequency refers to the collection cycle frequency of the sampling signal.

In this embodiment, body parameters refer to the target individual's body shape description parameters, such as height, weight, and specific indices.

In this embodiment, dynamic correction refers to correcting the current airway pressure signal based on the impact factor of the target individual's body shape on the compression force.

In this embodiment, continuous analysis refers to the continuous analysis of the target airway pressure signal in terms of time and frequency.

In this embodiment, the compression frequency, compression phase, and high-dimensional signal information for the target individual refer to the frequency and amplitude of chest compressions on the target individual.

In this embodiment, preset compression parameters and ventilation rule parameters refer to the corresponding rule parameters between the preset number of compressions and the timing of ventilation, for example, one ventilation after every 30 compressions.

In this embodiment, the mode setting parameters of the ventilation equipment refer to the ventilation setting parameters of the ventilation equipment in various working modes.

In this embodiment, ventilation probability and ventilation time window refer to the probability of the user receiving ventilation with each chest compression and the description duration window for confirming the number of chest compressions for ventilation.

The working principle of the above technical solution is as follows: obtain the current airway pressure signal when compressing the heart of the target individual at a preset sampling frequency; dynamically correct the current airway pressure signal based on the body parameters of the target individual to obtain the target airway pressure signal; continuously analyze the target airway pressure signal to obtain the compression frequency, compression phase, and high-dimensional signal information for the target individual; train a machine learning model with preset compression parameters and ventilation rule parameters; input the compression frequency, compression phase, and high-dimensional signal information for the target individual, along with the mode setting parameters of the ventilation equipment, into the machine learning model to determine the target pressure signal's ventilation probability and ventilation time window, and control the ventilation equipment to perform automatic ventilation on the target individual according to the ventilation time window.

The beneficial effects of the above technical solution are as follows: by automatically predicting the ventilation time window and performing automatic ventilation based on the compression frequency and compression phase of the target individual, the device can automatically perform cardiac compressions and timely ventilation on the target individual without the prerequisite of communication protocols. This reduces labor costs while also precisely controlling the timing of ventilation, improving safety, reliability, and stability. It avoids the influence of subjective factors from medical personnel, enhances objectivity, and solves the problem that existing coordination between the cardiopulmonary resuscitation compression and ventilation is usually managed by medical staff or the cardiopulmonary resuscitation compression equipment and ventilators are coordinated through communication protocols, which cannot be automatically coordinated and the manual coordination methods are difficult to precisely and stably control for long periods, which reduces practicality.

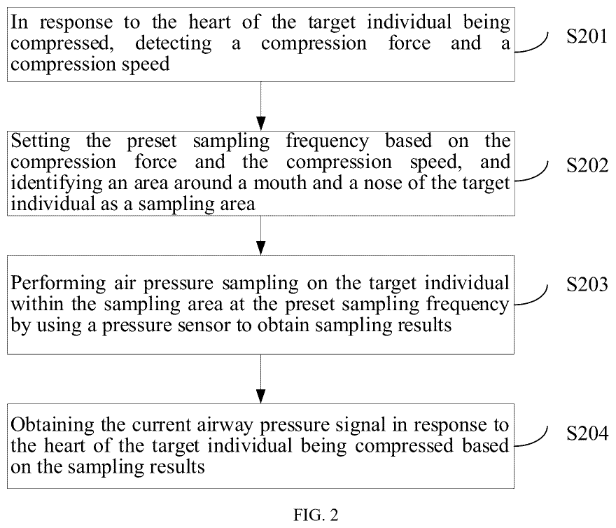

In an embodiment, as shown in FIG. 2, the obtaining a current airway pressure signal according to a preset sampling frequency in response to a heart of a target individual being comprised includes:
step S201, in response to the heart of the target individual being compressed, detecting the compression force and the compression speed;
step S202, setting the preset sampling frequency based on the compression force and speed, and identifying the area around the mouth and nose of the target individual as the sampling area;
step S203, performing air pressure sampling on the target individual within the sampling area at the preset sampling frequency by using a pressure sensor; and
step S204, obtaining the current airway pressure signal in response to the heart of the target individual being compressed based on the sampling results.

In this embodiment, the sampling area refers to the range area for signal sampling of the current airway pressure signal.

The beneficial effects of the above technical solution are as follows: by intelligently setting the sampling frequency, the sampling frequency can be reasonably set according to the real-time compression situation of the target individual, ensuring the high quality of the sampling data. Furthermore, by locating the sampling area, airflow signals and air pressure signals can be more accurately detected around the mouth and nose of the target individual, ensuring the precision of the detected signals.

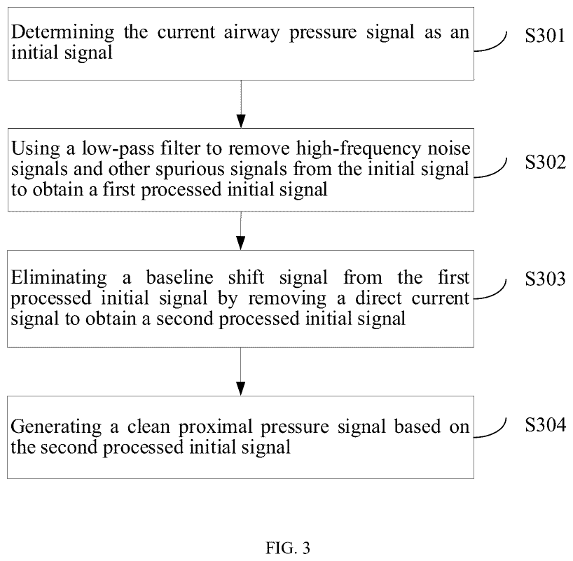

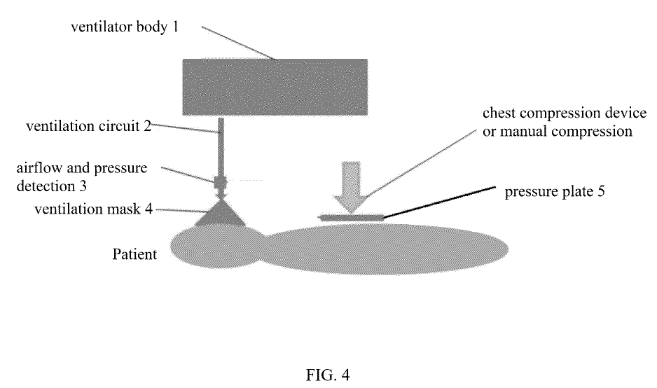

In an embodiment, as shown in FIG. 3, before continuously analyzing the current airway pressure signal to obtain the compression frequency, compression phase, and high-dimensional signal information for the target individual, the method further includes:
step S301, determining the current airway pressure signal as the initial signal;
step S302, using a low-pass filter to remove high-frequency noise signals and other spurious signals from the initial signal to obtain the first processed initial signal;
step S303, eliminating the baseline shift signal from the first processed initial signal by removing the direct current signal to obtain the second processed initial signal; and
step S304, generating a clean proximal pressure signal based on the second processed initial signal.

In this embodiment, the baseline shift signal refers to the target signal in the first processed initial signal where the signal baseline has shifted.

The beneficial effects of the above technical solution are as follows: it can ensure the purity of the signal and remove the influence of interference signals, laying the foundation for subsequent signal analysis and improving practicality.

In an embodiment, the dynamic correction of the current airway pressure signal based on the body parameters of the target individual to obtain the target airway pressure signal includes:
determining the current thoracic resonance parameters of the target individual based on the body parameters of the target individual;
determining the dynamic correction factor based on the current thoracic resonance parameters and the preset collection parameters of the current airway pressure signal;
using the dynamic correction factor to dynamically correct the current airway pressure signal; and
obtaining the target airway pressure signal based on the correction results.

In this embodiment, the current thoracic resonance parameters refer to the resonance description parameters between the chest cavity and the compression operation caused by the body shape of the target individual.

In this embodiment, the preset collection parameters refer to the collection frequency and collection intensity of the current airway pressure signal.

In this embodiment, the dynamic correction factor refers to the correction factor for a signal phase and a signal frequency of the current airway pressure signal.

The beneficial effects of the above technical solution are as follows: by determining the current thoracic resonance parameters based on the body parameters of the target individual and then correcting the collected airway pressure signal, the corrected airway pressure signal is more consistent with the target individual, improving the data referentiality.

In this embodiment, determining the dynamic correction factor based on the current thoracic resonance parameters and the preset collection parameters of the current airway pressure signal includes:
determining the set chest compression variation amplitude parameters based on the preset collection parameters of the current airway pressure signal;
determining the airway resistance index based on the chest compression variation amplitude parameters and the current thoracic resonance parameters;
generating a respiratory signal sample set using a respiratory mechanics model based on the airway resistance index;
determining the morphological parameter values at each point in the target individual's airway based on the respiratory signal sample set;
determining the range of the stress tensor in the target individual's airway based on the morphological parameter values at each point in the airway of the target individual;
acquiring characteristic parameters that maintain the steady-state performance of the target individual's airway, and obtaining the airway performance indicators of the target individual based on the characteristic parameters;
determining the stress differentiation in the target individual's airway based on the airway performance indicators and the range of the airway's stress tensor;
determining an error range between the standard data and actual detection data of a chest compression force of the target individual based on the stress differentiation; and
determining the dynamic correction factor for the current airway pressure signal based on the error range between the standard data and actual detection data of the chest compression force of the target individual, and the detected pressure data corresponding to the current airway pressure signal.

In this embodiment, the set chest compression variation amplitude parameters refer to the amplitude data parameters of the chest cavity fluctuations with compression under set conditions for the target individual.

In this embodiment, the airway resistance index refers to the index of ventilation resistance difficulty in the airway of the target individual under body shape compression.

In this embodiment, the respiratory mechanics model is represented as a network model used for simulating respiratory mechanics.

In this embodiment, the respiratory signal sample set represents a dataset of respiratory signal samples simulated under the airway resistance index through the respiratory mechanics model.

In this embodiment, the morphological parameter values at various points in the target individual's airway represent the parameter values of the undulating morphology at each distributed sampling point in the target individual's airway.

In this embodiment, the range of the stress tensor represents the numerical range of the tensor in the target individual's airway after being subjected to compression force.

In this embodiment, the characteristic parameters that maintain the steady-state performance of the target individual's airway represent the relevant characteristic parameters used to maintain the steady-state performance of the target individual's airway.

In this embodiment, the airway performance indicators represent the indicators for maintaining good respiratory performance of the airway.

In this embodiment, the stress differentiation refers to the differentiation and loss of feedback airflow in the target individual's airway when undergoing chest compressions.

The beneficial effects of the above technical solution are as follows: by determining the airway obstruction parameters of the target individual and then determining the stress differentiation of the target individual's airway during compression, the conversion relationship between the actual compression force and the stress force can be accurately assessed to determine the dynamic correction factor for the detected signal, ensuring the accuracy and objectivity of the final dynamic correction factor.

In an embodiment, continuously analyzing the current airway pressure signal to obtain the compression frequency, compression phase, and high-dimensional signal information for the target individual includes:
analyzing the clean proximal pressure signal by using a time-domain analysis algorithm to obtain the first analysis result;
analyzing the clean proximal pressure signal by using a frequency-domain analysis algorithm to obtain the second analysis result;
determining the compression frequency, compression interval, and duration of a single compression for the target individual based on the first analysis result, and determining the compression phase based on the compression interval and duration of a single compression; and
obtaining the frequency-domain characteristics of the clean proximal pressure signal based on the second analysis result, and performing a deep analysis on the frequency-domain characteristics of the clean proximal pressure signal to obtain high-dimensional signal information.

The beneficial effects of the above technical solution are as follows: by performing dual analysis, the detailed high-dimensional information such as the compression-related rule parameters and compression intensity for the target individual can be accurately analyzed, laying the foundation for subsequent model training and further improving practicality.

In an embodiment, analyzing the proximal pressure signal using a frequency-domain analysis algorithm to obtain the second analysis result includes:
performing a time-frequency conversion on the proximal pressure signal to by using a window function and Fourier transform to obtain a spectral signal;
marking the peak of the spectral signal, capturing areas of region around the peak, as well as the low-frequency region and high-frequency region of the spectral signal;
obtaining the regional signals respectively corresponding to the areas of the region around the peak, and the low-frequency region and high-frequency region; and
performing a geometric calculation on the regional signals respectively corresponding to the areas of the region around the peak, and the low-frequency region and high-frequency region using a frequency-domain analysis algorithm to obtain the second analysis result.

The beneficial effects of the above technical solution are as follows: it maximizes the acquisition of the frequency-domain analysis results of the signal, ensuring the completeness and reliability of the high-dimensional information.

In an embodiment, training the machine learning model with preset compression parameters and ventilation rule parameters includes:
generating compression pattern parameters based on a preset machine learning algorithm and the preset compression parameters;
generating the first training sample based on the compression pattern parameters, and setting model learning parameters and model output parameters based on ventilation rule parameters; and
training the machine learning model with the first training sample based on the model learning parameters and model output parameters.

The beneficial effects of the above technical solution are as follows: by generating training samples and simultaneously setting model learning parameters and output parameters, stable convergence can be achieved during the model training process, ensuring the subsequent stability of model recognition, and improving practicality and reliability.

In an embodiment, inputting the target individual's compression frequency, compression phase, and high-dimensional signal information, along with the mode setting parameters of the ventilation equipment into the machine learning model to determine the target pressure signal's ventilation probability and ventilation time window, and controlling the ventilation equipment to perform automatic ventilation on the target individual according to the ventilation time window, includes:
obtaining the ventilation compression ratio, target frequency, and ventilation threshold for each of different ventilation modes based on the mode setting parameters of the ventilation equipment;
determining the target ventilation mode from the different ventilation modes based on the target individual's compression frequency and the ventilation compression ratio, target frequency, and ventilation threshold for each of the different ventilation modes;
acquiring the ventilation compression phase under the target ventilation mode, and matching the ventilation compression phase based on the target individual's compression phase using a dynamic threshold method;
determining the ventilation probability of the target airway pressure signal based on the matched results, and determining the ventilation time window threshold based on the position relationship between the ventilation compression phase and the target individual's compression phase; and
controlling the ventilation equipment to perform automatic ventilation on the target individual according to the ventilation time window threshold and the ventilation threshold.

The beneficial effects of the above technical solution are as follows: it can both grasp the timing of ventilation for the target individual and control the amount of ventilation, further ensuring the safety and the probability of successful resuscitation of the target individual.

In an embodiment, this embodiment also discloses a ventilator suitable for the above cardiopulmonary resuscitation airway pressure automatic feedback ventilation method, the device includes: a ventilator body 1, a ventilation circuit 2, airflow and pressure detection equipment 3, a ventilation mask 4, and a pressure plate 5;

The pressure plate 5 is used to perform artificial or mechanical chest compressions on the target individual;

The ventilation mask 4 is used to cover the mouth and nose area of the target individual;

The airflow and pressure detection equipment 3 is used to detect the airway pressure signal of the target individual's heart during compression;

The ventilator body 1 is used to analyze the airway pressure signal, obtain information about the target individual's cardiopulmonary resuscitation compressions, and based on this information, determine the ventilation time window, issue ventilation commands, and perform a ventilation operation;

The ventilation circuit 2 is used to input the ventilating agent from the ventilator body into the mouth and nose of the target individual.

The working principle and beneficial effects of the above technical solution have been explained in the method claims and are not repeated here.

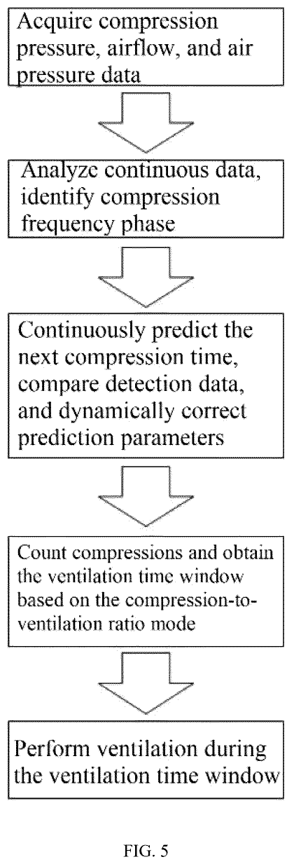

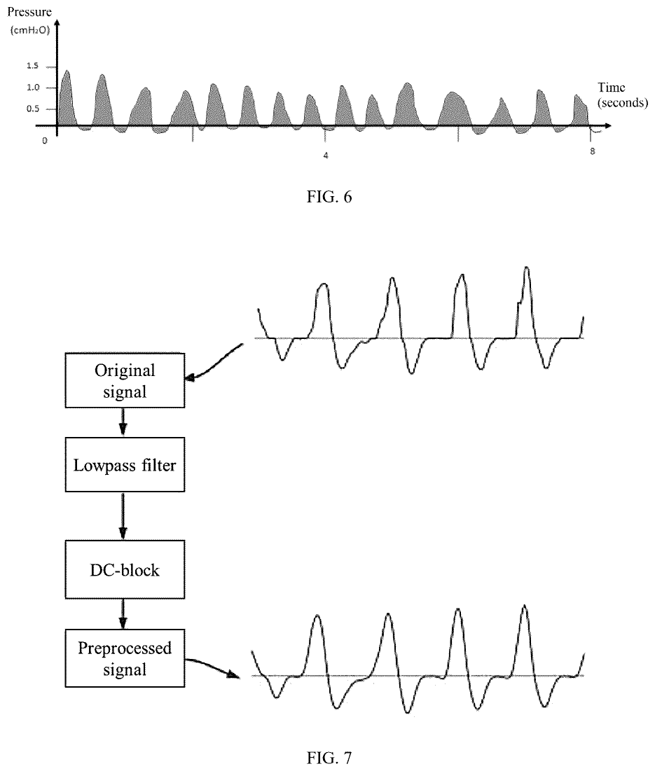

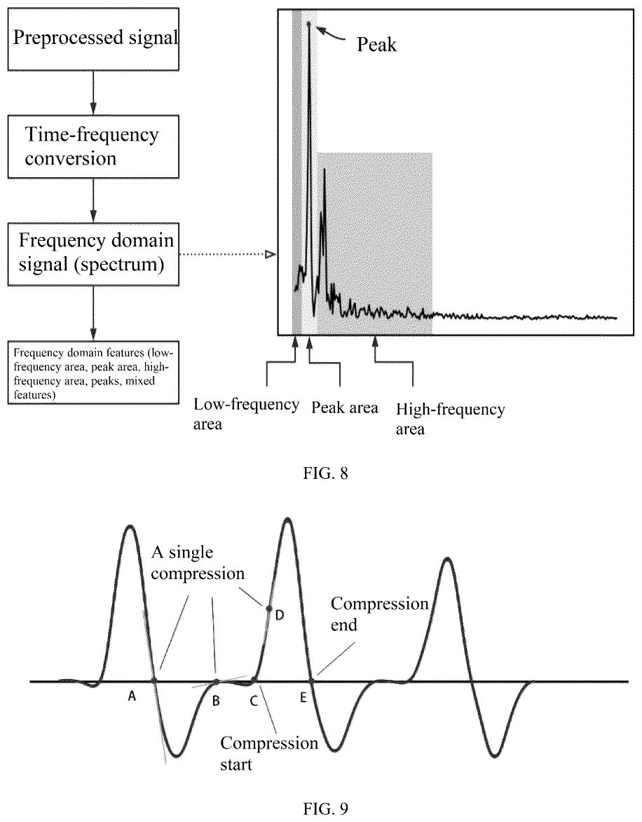

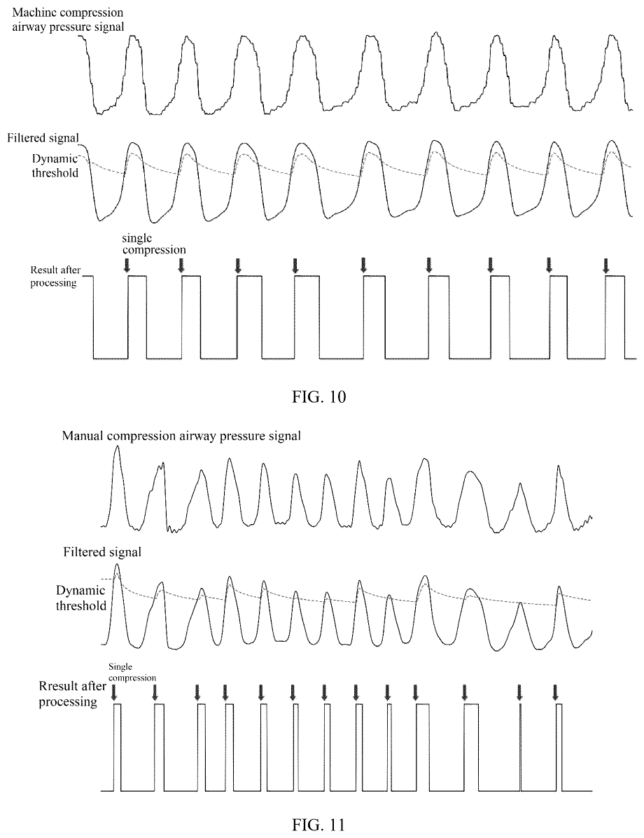

In an embodiment, as shown in FIG. 5, by analyzing the compression pressure and airflow data, the airway pressure curve during the cardiac compression process is obtained, which is as shown in FIG. 6. First, as shown in FIG. 7, the signal is preprocessed, using a low-pass filter to remove high-frequency noise (such as equipment vibration, etc.) and other spurious signals (such as electromagnetic interference, etc.) contained in the original signal. Then, by eliminating the direct current signal, the baseline shift signal (such as the positive end-expiratory pressure abbreviated as PEEP setting of the respiratory equipment, etc.) is removed to obtain a clean proximal pressure signal. Next, as shown in FIG. 8 and FIG. 9, the signal is analyzed in the frequency-domain and time-domain. The time-domain and frequency-domain features obtained, combined with the mode settings of the ventilation equipment, are given to the machine learning model for judgment, to determine whether ventilation is needed, and to control the ventilation device (i.e., the ventilation equipment) to perform ventilation. As shown in FIG. 10 and FIG. 11, the timing of ventilation for machine compression and manual compression is determined by a dynamic threshold method.

Those skilled in the technical field will readily think of other embodiments of this disclosure after considering the specification and practicing the disclosure herein. This application is intended to cover any variations, uses, or adaptations of the disclosure that follow the general principles of the disclosure and include means of common knowledge or customary skill in the art not disclosed herein. The specification and embodiments are to be regarded as exemplary only, and the true scope and spirit of the present disclosure is indicated by the following claims.

It should be understood that the present disclosure is not limited to the precise structure that has been described above and illustrated in the accompanying drawings, and that various modifications and changes may be made without departing from its scope. The scope of the present disclosure is limited only by the appended claims.

What is claimed is:

1. A cardiopulmonary resuscitation airway pressure automatic feedback ventilation method, comprising:
obtaining a current airway pressure signal according to a preset sampling frequency in response to a heart of a target individual being compressed;
dynamically correcting the current airway pressure signal based on body parameters of the target individual to obtain a target airway pressure signal;
continuously analyzing the target airway pressure signal to obtain a compression frequency, a compression phase, and signal information for the target individual;
training a machine learning model according to preset compression parameters and ventilation rule parameters; and
inputting the compression frequency, the compression phase and the signal information for the target individual, and mode setting parameters of a ventilation equipment, into the machine learning model to determine a ventilation probability and a ventilation time window of the target airway pressure signal, and controlling the ventilation equipment to perform automatic ventilation on the target individual according to the ventilation time window and the ventilation probability.

2. The cardiopulmonary resuscitation airway pressure automatic feedback ventilation method according to claim 1, wherein the obtaining a current airway pressure signal according to a preset sampling frequency in response to a heart of a target individual being comprised comprises:
in response to the heart of the target individual being compressed, detecting a compression force and a compression speed;
setting the preset sampling frequency based on the compression force and the compression speed, and identifying an area around a mouth and a nose of the target individual as a sampling area;
performing air pressure sampling on the target individual within the sampling area at the preset sampling frequency by using a pressure sensor to obtain sampling results; and
obtaining the current airway pressure signal in response to the heart of the target individual being compressed based on the sampling results.

3. The cardiopulmonary resuscitation airway pressure automatic feedback ventilation method according to claim 1, wherein before the continuously analyzing the current airway pressure signal to obtain a compression frequency, a compression phase, and signal information for the target individual, the method further comprises:
determining the current airway pressure signal as an initial signal;
using a low-pass filter to remove high-frequency noise signals and other spurious signals from the initial signal to obtain a first processed initial signal;
eliminating a baseline shift signal from the first processed initial signal by removing a direct current signal to obtain a second processed initial signal; and
generating a clean proximal pressure signal based on the second processed initial signal.

4. The cardiopulmonary resuscitation airway pressure automatic feedback ventilation method according to claim 1, wherein the dynamically correcting the current airway pressure signal based on body parameters of the target individual to obtain a target airway pressure signal, comprises:
determining current thoracic resonance parameters of the target individual based on the body parameters of the target individual;
determining a dynamic correction factor based on the current thoracic resonance parameters and preset collection parameters of the current airway pressure signal;
using the dynamic correction factor to dynamically correct the current airway pressure signal to obtain correction results; and
obtaining the target airway pressure signal based on the correction results.

5. The cardiopulmonary resuscitation airway pressure automatic feedback ventilation method according to claim 3, wherein the continuously analyzing the current airway pressure signal to obtain a compression frequency, a compression phase, and signal information for the target individual, comprises:
analyzing the clean proximal pressure signal by using a time-domain analysis algorithm to obtain a first analysis result;
analyzing the clean proximal pressure signal by using a frequency-domain analysis algorithm to obtain a second analysis result;
determining the compression frequency, a compression interval, and a duration of a single compression for the target individual based on the first analysis result, and determining the compression phase based on the compression interval and the duration of the single compression; and
obtaining frequency-domain characteristics of the clean proximal pressure signal based on the second analysis result, and performing a deep analysis on the frequency-domain characteristics of the clean proximal pressure signal to obtain the signal information.

6. The cardiopulmonary resuscitation airway pressure automatic feedback ventilation method according to claim 5, wherein the analyzing the proximal pressure signal by using a frequency-domain analysis algorithm to obtain a second analysis result comprises:
performing a time-frequency conversion on the proximal pressure signal by using a window function and a Fourier transform to obtain a spectral signal;
marking a peak of the spectral signal, and capturing areas of region around the peak, a low-frequency region and a high-frequency region of the spectral signal;
obtaining regional signals respectively corresponding to the areas of the region around the peak, and the low-frequency region and the high-frequency region; and
performing a geometric calculation on the regional signals respectively corresponding to the areas of the region around the peak, and the low-frequency region and the high-frequency region by using a frequency-domain analysis algorithm to obtain the second analysis result.

7. The cardiopulmonary resuscitation airway pressure automatic feedback ventilation method according to claim 1, wherein the training a machine learning model according to preset compression parameters and ventilation rule parameters comprises:
generating compression pattern parameters based on a preset machine learning algorithm and the preset compression parameters;
generating a first training sample based on the compression pattern parameters, and setting model learning parameters and model output parameters based on the ventilation rule parameters; and
training the machine learning model with the first training sample based on the model learning parameters and the model output parameters.

8. The cardiopulmonary resuscitation airway pressure automatic feedback ventilation method according to claim 1, wherein the inputting the compression frequency, the compression phase, and the signal information for the target individual, and mode setting parameters of ventilation equipment, into the machine learning model to determine a ventilation probability and a ventilation time window of the target airway pressure signal, and controlling the ventilation equipment to perform automatic ventilation on the target individual according to the ventilation time window and the ventilation probability, comprises:
obtaining a ventilation compression ratio, a target frequency, and a ventilation threshold for each of different ventilation modes based on the mode setting parameters of the ventilation equipment;
determining a target ventilation mode from the different ventilation modes based on the compression frequency of the target individual and the ventilation compression ratio, the target frequency and the ventilation threshold for each of the different ventilation modes;
acquiring a ventilation compression phase under the target ventilation mode, and matching the ventilation compression phase based on the compression phase of the target individual using a dynamic threshold method to obtain matched results;
determining the ventilation probability of the target airway pressure signal based on the matched results, and determining a threshold of the ventilation time window based on a position relationship between the ventilation compression phase and the compression phase of the target individual; and
controlling the ventilation equipment to perform automatic ventilation on the target individual according to the threshold of the ventilation time window, the ventilation probability and the ventilation threshold.

9. The cardiopulmonary resuscitation airway pressure automatic feedback ventilation method according to claim 4, wherein the determining a dynamic correction factor based on the current thoracic resonance parameters and preset collection parameters of the current airway pressure signal comprises:
determining chest compression variation amplitude parameters based on the preset collection parameters of the current airway pressure signal;
determining airway resistance index based on the chest compression variation amplitude parameters and the current thoracic resonance parameters;
generating a respiratory signal sample set using a respiratory mechanics model based on the airway resistance index;
determining morphological parameter values at each point in an airway of the target individual based on the respiratory signal sample set;
determining a range of a stress tensor in the airway of the target individual based on the morphological parameter values at each point in the airway of the target individual;
acquiring characteristic parameters that maintain steady-state performance of the airway of the target individual, and obtaining airway performance indicators of the target individual based on the characteristic parameters;
determining stress differentiation in the airway of the target individual based on the airway performance indicators and the range of stress tensor of the airway of the target individual;
determining an error range between standard data and actual detection data of a chest compression force of the target individual based on the stress differentiation; and
determining the dynamic correction factor for the current airway pressure signal based on the error range between the standard data and the actual detection data of the chest compression force of the target individual, and detected pressure data corresponding to the current airway pressure signal.

10. The cardiopulmonary resuscitation airway pressure automatic feedback ventilation method according to claim 1, wherein the preset sampling frequency is a collection cycle frequency for a sampling signal.

11. The cardiopulmonary resuscitation airway pressure automatic feedback ventilation method according to claim 1, wherein the body parameters are body shape description parameters of the target individual.

12. The cardiopulmonary resuscitation airway pressure automatic feedback ventilation method according to claim 1, wherein the preset compression parameters and the ventilation rule parameters are represented as a rule parameter corresponding to a preset number of compressions and a ventilation timing.

13. The cardiopulmonary resuscitation airway pressure automatic feedback ventilation method according to claim 2, wherein the sampling area is an area for signal sampling of the current airway pressure signal.

14. The cardiopulmonary resuscitation airway pressure automatic feedback ventilation method according to claim 3, wherein the baseline shift signal is a target signal with a signal baseline offset in the first processed initial signal.

15. The cardiopulmonary resuscitation airway pressure automatic feedback ventilation method according to claim 4, wherein the preset collection parameters comprise a collection frequency and a collection intensity of the current airway pressure signal.

16. The cardiopulmonary resuscitation airway pressure automatic feedback ventilation method according to claim 4, wherein the dynamic correction factor is a correction factor between a signal phase and a signal frequency of the current airway pressure signal.

17. The cardiopulmonary resuscitation airway pressure automatic feedback ventilation method according to claim 9, wherein the airway resistance index is an index of ventilation resistance difficulty in an airway of the target individual under a body shape compression.

18. The cardiopulmonary resuscitation airway pressure automatic feedback ventilation method according to claim 9, wherein the respiratory mechanics model is a network model, configured to simulate respiratory mechanics.

19. A ventilator, applied for the cardiopulmonary resuscitation airway pressure automatic feedback ventilation method according to claim 1, wherein the ventilator comprises: a ventilator body, a ventilation circuit, an airflow and pressure detection equipment, a ventilation mask, and a pressure plate;
wherein the pressure plate is configured to perform artificial or mechanical chest compressions on the target individual;
wherein the ventilation mask is configured to cover a mouth and nose area of the target individual;
wherein the airflow and pressure detection equipment is configured to detect an airway pressure signal of the heart of the target individual during compression;
wherein the ventilator body is configured to analyze the airway pressure signal, obtain information about cardiopulmonary resuscitation compressions of the target individual, analyze the information about cardiopulmonary resuscitation compressions of the target individual to determine the ventilation time window, issue a ventilation command, and perform a ventilation operation; and
wherein the ventilation circuit is configured to input gas to be ventilated from the ventilator body into the mouth and nose of the target individual.

20. A ventilator, comprising:
a ventilator body;
a ventilation circuit, connected to the ventilator body; wherein the ventilation circuit is configured to input gas to be ventilated from the ventilator body into a mouth and a nose of a target individual;
an airflow and air pressure detection equipment, connected to the ventilation circuit; wherein the airflow and air pressure detection equipment is configured to detect an airway pressure signal of a heart of the target individual during compression;
a ventilation mask, connected to the airflow and air pressure detection equipment; wherein the ventilation mask is configured to cover the mouth and the nose of the target individual; and
a pressure plate, configured to perform artificial or mechanical chest compressions on the target individual; and
wherein the ventilator body is configured to analyze an airway pressure signal, obtain information about cardiopulmonary resuscitation compressions of the target individual, determine a ventilation time window based on the information about cardiopulmonary resuscitation compressions of the target individual, issue a ventilation command, and perform a ventilation operation.
